(19) United States
(12) Patent Application Publication
(10) Pub. No.: US 2017/0143803 A1
(43) Pub. Date: May 25, 2017

(54) MODIFICATION OF PHARMACEUTICAL PREPARATIONS TO MAKE THEM MORE CONDUCIVE TO ULTRASONIC TRANSDERMAL DELIVERY

(71) Applicant: BKR IP Holdco LLC, Broomall, PA (US)

(72) Inventor: Bruce K. REDDING, Jr., Broomall, PA (US)

(21) Appl. No.: 15/323,636

(22) PCT Filed: Jul. 6, 2015
(86) PCT No.: PCT/US2015/039831

§ 371 Date: Jan. 3, 2017

Related U.S. Application Data
(60) Provisional application No. 61/998,622, filed on Jul. 3, 2014.
(60) Provisional application No. 61/998,623, filed on Jul. 3, 2014.
(60) Provisional application No. 61/998,624, filed on Jul. 3, 2014.
(60) Provisional application No. 61/998,683, filed on Jul. 7, 2014.
(60) Provisional application No. 61/998,788, filed on Jul. 9, 2014.
(60) Provisional application No. 61/998,790, filed on Jul. 9, 2014.
(60) Provisional application No. 61/999,589, filed on Aug. 1, 2014.
(60) Provisional application No. 62/125,837, filed on Feb. 2, 2015.

(30) Foreign Application Priority Data
Jul. 6, 2015 (US) PCT US2015/039236
Jul. 6, 2015 (US) PCT US2015/039264
Jul. 6, 2015 (US) PCT US2015/039268
Jul. 6, 2015 (US) PCT US2015/039272

(51) Int. Cl. A61K 38/28; A61M 37/00; A61K 9/70; A61K 9/00; A61K 41/00

(52) U.S. Cl. CPC A61K 38/28; A61K 9/0014; A61K 41/0047; A61K 9/0009; A61K 9/703; A61M 37/0092; A61M 2037/0007; A61M 2210/083; A61M 2210/10

(57) ABSTRACT

A method for improving the ultrasonic transdermal delivery of an drug by modifying the excipient solution to which an active ingredient is intermixed in a drug formulation, whereby the choice of excipient solution is modified to one which will be more conducive to ultrasound and will propagate the drug substance at a higher delivery speed through the skin under ultrasonic excitation An example of such an excipient change includes a conversion from a standard dibasic sodium phosphate containing formulation to one using far less sodium or less preservative compositions. Reduced dibasic sodium phosphate formulation. Responsively to insonification thereof, including: reducing the amount of dibasic sodium phosphate in the formulation to provide a reduced dibasic sodium phosphate formulation; and, making a substance in accordance with the reduced dibasic sodium phosphate formulation.

### PRIORITY CLAIM, CROSS REFERENCE TO RELATED APPLICATIONS, INCORPORATION BY REFERENCE AND CONTINUATION-IN-PART

This application is related to, claims priority under, and claims the benefit of the following provisional applications filed in the United States Patent and Trademark Office: “MODIFIED TRANSDERMAL DELIVERY PATCH WITH MULTIPLE ABSORBENT PADS”, Bruce K. Redding, Jr., filed on Jul. 3, 2014, and having Ser. No. 61/998,623: “MODIFIED TRANSDERMAL DELIVERY DEVICE OR PATCH AND METHOD OF DELIVERING INSULIN FROM SAID MODIFIED TRANSDERMAL DELIVERY DEVICE”, Bruce K. Redding, Jr., filed on Jul. 3, 2014, and having Ser. No. 61/998,622; “METHOD FOR GLUCOSE CONTROL IN DIABETICS”, Bruce K. Redding, Jr., filed on Jul. 3, 2014, and having Ser. No. 61/998,624; “ULTRASONIC TRANSDUCERS SUITABLE FOR ULTRASONIC DRUG DELIVERY VIA A SYSTEM WHICH IS PORTABLE AND WEARABLE BY THE PATIENT”, Bruce K. Redding, Jr., filed on Jul. 7, 2014, and having Ser. No. 61/998,683; “METHOD FOR THE ATTENUATION ENHANCEMENT OF ABSORBENT MATERIALS USED IN BOTH PASSIVE AND ACTIVE TRANSDERMAL DRUG DELIVERY SYSTEMS”, Bruce K. Redding, Jr., filed on Jul. 9, 2014, and having Ser. No. 61/998,788; “MODIFICATION OF PHARMACEUTICAL PREPARATIONS TO MAKE THEM MORE CONDUCIVE TO ULTRASONIC TRANSDERMAL DELIVERY”, Bruce K. Redding, Jr., filed on Jul. 9, 2014, and having Ser. No. 61/998,790; “METHOD AND APPARATUS FOR MEASURING THE DOSE REMAINING UPON A TRANSDERMAL DRUG DELIVERY DEVICE”, Bruce K. Redding, Jr., filed on Aug. 1, 2014, and having Ser. No. 61/999,589; “METHOD AND APPARATUS FOR EFFECTING ALTERNATING ULTRASONIC TRANSMISSIONS WITHOUT CAVITATION”, Bruce K. Redding, Jr., filed on Feb. 2, 2015, and having Ser. No. 62/125,837; PCT applications filed in the United States Patent and Trademark Office: “MODIFIED TRANSDERMAL DELIVERY PATCH WITH MULTIPLE ABSORBENT PADS”, Bruce K. Redding, Jr., filed on Jul. 6, 2015, and having serial number PCT/US15/39236; MODIFIED TRANSDERMAL DELIVERY DEVICE OR PATCH AND METHOD OF DELIVERING INSULIN FROM SAID MODIFIED TRANSDERMAL DELIVERY DEVICE, Bruce K. Redding, Jr., filed on Jul. 6, 2015, and having serial number PCT/US15/39264; METHOD FOR GLUCOSE CONTROL IN DIABETICS Bruce K. Redding, Jr., filed on Jul. 6, 2015, PCT/US15/39268; MODIFICATION OF PHARMACEUTICAL PREPARATIONS TO MAKE THEM MORE CONDUCIVE TO ULTRASONIC TRANSDERMAL DELIVERY, Bruce K. Redding, Jr., filed on Jul. 6, 2015, PCT/US15/39272.

This application hereby incorporates herein by reference the subject matter disclosed in the written descriptions, abstracts, the drawings and claims, in their entireties of the following provisional applications filed in the United States Patent and Trademark Office: “MODIFIED TRANSDERMAL DELIVERY PATCH WITH MULTIPLE ABSORBENT PADS”, Bruce K. Redding, Jr., filed on Jul. 3, 2014, and having Ser. No. 61/998,623: “MODIFIED TRANSDERMAL DELIVERY DEVICE OR PATCH AND METHOD OF DELIVERING INSULIN FROM SAID MODIFIED TRANSDERMAL DELIVERY DEVICE”, Bruce K. Redding, Jr., filed on Jul. 3, 2014, and having Ser. No. 61/998,622; “METHOD FOR GLUCOSE CONTROL IN DIABETICS”, Bruce K. Redding, Jr., filed on Jul. 3, 2014, and having Ser. No. 61/998,624; “ULTRASONIC TRANSDUCERS SUITABLE FOR ULTRASONIC DRUG DELIVERY VIA A SYSTEM WHICH IS PORTABLE AND WEARABLE BY THE PATIENT”, Bruce K. Redding, Jr., filed on Jul. 7, 2014, and having Ser. No. 61/998,683; “METHOD FOR THE ATTENUATION ENHANCEMENT OF ABSORBENT MATERIALS USED IN BOTH PASSIVE AND ACTIVE TRANSDERMAL DRUG DELIVERY SYSTEMS”, Bruce K. Redding, Jr., filed on Jul. 9, 2014, and having Ser. No. 61/998,788; “MODIFICATION OF PHARMACEUTICAL PREPARATIONS TO MAKE THEM MORE CONDUCIVE TO ULTRASONIC TRANSDERMAL DELIVERY”, Bruce K. Redding, Jr., filed on Jul. 9, 2014, and having Ser. No. 61/998,790; “METHOD AND APPARATUS FOR MEASURING THE DOSE REMAINING UPON A TRANSDERMAL DRUG DELIVERY DEVICE”, Bruce K. Redding, Jr., filed on Aug. 1, 2014, and having Ser. No. 61/999,589; “METHOD AND APPARATUS FOR EFFECTING ALTERNATING ULTRASONIC TRANSMISSIONS WITHOUT CAVITATION”, Bruce K. Redding, Jr., filed on Feb. 2, 2015, and having Ser. No. 62/125,837; PCT applications filed in the United States Patent and Trademark Office: “MODIFIED TRANSDERMAL DELIVERY PATCH WITH MULTIPLE ABSORBENT PADS”, Bruce K. Redding, Jr., filed on Jul. 6, 2015, and having serial number PCT/US15/39236; MODIFIED TRANSDERMAL DELIVERY DEVICE OR PATCH AND METHOD OF DELIVERING INSULIN FROM SAID MODIFIED TRANSDERMAL DELIVERY DEVICE, Bruce K. Redding, Jr., filed on Jul. 6, 2015, and having serial number PCT/US15/39264; METHOD FOR GLUCOSE CONTROL IN DIABETICS Bruce K. Redding, Jr., filed on Jul. 6, 2015, PCT/US15/39268.

This application is a continuation-in-part of co-pending non-provisional application entitled MODIFICATION OF PHARMACEUTICAL PREPARATIONS TO MAKE THEM MORE CONDUCIVE TO ULTRASONIC TRANSDERMAL DELIVERY, by Bruce K. Redding, Jr., filed on Jul. 6, 2015, PCT/US15/39272.

### FIELD OF THE INVENTION

The present invention relates generally to substance delivery methods, and more particularly to methods suitable for enhancing dermal and transdermal substance delivery.

### BACKGROUND OF THE INVENTION

Generally, transdermal medicinal compound, or drug, delivery systems employ a medicated patch, which is affixed to the skin of a patient. The patch allows the medicinal compound to be absorbed through the skin layers and into the patient's blood stream. Transdermal drug delivery generally mitigates the pain associated with injections and intravenous administration, as well as the risk of infection associated with these techniques. Transdermal drug delivery also avoids gastrointestinal metabolism of administered drugs, mitigates liver metabolization thereof, and provides a sustained release of the administered drug. Transdermal drug delivery also enhances patient compliance with a drug regimen, because of the relative ease of administration and the sustained release of the drug.

Many drugs, however, are not well suited for administration via conventional transdermal drug delivery systems. For example, some substances are absorbed with difficulty through the skin due to molecular size or bio-adhesion properties. In these cases, when transdermal drug delivery is attempted, the drug may pool on the outer surface of the skin, not permeating through the skin into the blood stream. An example of such a drug is insulin, which has been found difficult to administer via conventional transdermal drug delivery systems.

Consistently, conventional transdermal drug delivery methods have been found suitable only for low molecular weight medications—such as nitroglycerin for alleviating angina, nicotine for smoking cessation regimens, and estradiol for estrogen replacement in post-menopausal women. Larger molecular medications such as insulin (a polypeptide for the treatment of diabetes), erythropoietin (used to treat severe anemia) and gamma-interferon (used to boost the immune systems cancer fighting ability) are all compounds not normally effective when used with conventional transdermal drug delivery methods.

One method of assisting transdermal drug delivery is by iontophoresis. Iontophoresis involves the application of an external electric field and topical delivery of an ionized form of drug or unionized drug carried with the water flux associated with ion transport (electro-osmosis). Iontophoresis has been proposed to increase the permeability of skin to drugs. While permeation enhancement with iontophoresis may be effective, control of drug delivery and irreversible skin damage are problems associated with the technique.

Ultrasound has also been suggested to enhance permeability of the skin. Ultrasonic signals can be generated by vibrating a piezoelectric crystal or other electromechanical element, such as by passing an alternating current there through. The use of ultrasound to increase the permeability of the skin to drug molecules is sometimes referred to as sonophoresis or phonophoresis. However, while the use of conventional ultrasound for drug delivery has been generally suggested, results have been largely disappointing—in that enhancement of skin permeability has been relatively low. This is largely due to the use of sine wave ultrasound to breach the skin cavities and to drive the drug into the skin. Further, it is believed that there is no consensus on the efficacy of ultrasound for increasing drug flux across the skin. While some studies report the success of sonophoresis, others have obtained negative results.

The use of ultrasound coupled with an alternating ultrasonic transmission, one which alternates between one waveform and then switches to another waveform has been found to avoid cavitation and heat energy associated with conventional sinusoidal ultrasound, and to be an effective drug delivery mechanism. U.S. Pat. No. 7,440,798, Substance Delivery Device, Bruce K. Redding, Jr. inventor, granted Oct. 21, 2008, discloses the use of an alternating ultrasound system for drug delivery.

In view of the foregoing, safely enhancing drug transport across the skin is desirable.

### SUMMARY OF THE INVENTION

A method for improving the ultrasonic transdermal delivery of an drug by modifying the excipient solution to which an active ingredient is intermixed in a drug formulation, whereby the choice of excipient solution is modified to one which will be more conducive to ultrasound and will propagate the drug substance at a higher delivery speed through the skin under ultrasonic excitation

### BRIEF DESCRIPTION OF THE FIGURES

Understanding of the present invention will be facilitated by consideration of the following detailed description of the preferred embodiments of the present invention taken in conjunction with the accompanying drawings, in which like numerals refer to like parts and in which:

FIG. 1 illustrates an absorbent pad incorporating patch suitable for use with ultrasonic signals to deliver a substance transdermally into a patient as disclosed in U.S. Pat. No. 7,440,798, Substance Delivery Device, Bruce K. Redding, Jr.; and,

FIG. 2 illustrates a chart indicative of different transdermal delivery rates for different commercially available forms of insulin.

FIG. 3 is an illustration of the structure of human skin.

FIG. 4 is an illustration of a waist mounted ultrasonic drug delivery system.

FIG. 5 is an illustration of an arm mounted ultrasonic drug delivery system.

FIG. 6 is a design for the Franz cell used in Experiment-1

FIG. 7 is a single element transducer design.

FIG. 8 in an array consisting of 9 transducer elements.

FIG. 9 in an array consisting of 4 transducer elements affixed to a stainless steel face plate.

FIG. 10 is an alternating ultrasonic transmission.

FIG. 11 is the same as FIG. 2 but shows the uniformity of power intensity, ultrasonic frequency and whether Humulin or Humalog was used along with which type of transducer array.

FIGS. 12 and 13 show the differences in delivery speed for differing types of insulin via a standard ultrasonic excitation.

### DETAILED DESCRIPTION OF THE INVENTION

It is to be understood that the figures and descriptions of the present invention have been simplified to illustrate elements that are relevant for a clear understanding of the present invention, while eliminating, for purposes of clarity, many other elements found in typical pharmaceutical preparations, as wells as transdermal delivery methods and systems. However, because such elements are well known in the art, and because they do not facilitate a better understanding of the present invention, a discussion of such elements is not provided herein. The disclosure herein is directed to all such variations and modifications known to those skilled in the art.

Applicant believes large molecular formulations can be effectively delivered responsively to ultrasound application or insonification. FIG. 1 illustrates a transdermal patch 100 suitable for use with ultrasonic signals, wherein the patch employs at least one absorbent pad or layer of absorbent material. Patch 100 is constructed with a backbone or backing material 10 into which a section, or aperture, has been created. In the illustrated embodiment, the aperture accommodates a sonic membrane 11. A peel-away film 12 seals patch 300 until use. Peel-away film 12 may be constructed of any suitable material, including, but not limited to, UV-resistant, anti-static polyethylene film (50 micrometer thickness) available from Crystal-X Corp., of Sharon Hill, Pa. In the illustrated embodiment, and oppositely disposed from membrane 11, is a semi-permeable member 13. Member 13 may take the form of a membrane or film that comes into functional proximity with the skin of a user. For example, the patch may be adhered to the skin such that membrane 11 is in direct contact with the skin. In the interior of patch 100 is an absorbent pad 14 that holds a desired substance, e.g., drug or medicinal compound 15. In the illustrated embodiment, a gasket 16 is placed between backbone 10 and absorbent pad 14. Gasket 16 may be composed of any suitable material, such as, for example, synthetic rubber. Gasket 16 forms a reservoir or well over which absorbent pad 14 is placed. When pressed upon the skin, gasket 16 forms a barrier, which tends to restrict moisture and air from traveling under the patch and interfering with the ultrasonic signal intensity. Alternatively, a sealant compound, ultrasonic gel or other suitable material may be used for or in place of the gasket 16 to provide a sealing action around the borders of patch 100 to provide moisture protection, prevent leakage of substance or the drug from the patch and prevent air from entering under the patch. Of course, numerous changes to the components or construction of patch 100 may be made without departing from the spirit of the invention disclosed herein. In addition, in a similar manner ultrasound and other forms of external stimuli may be used in addition to or in lieu of ultrasound on delivery devices other than transdermal patches, including but not limited to bandages.

An external stimulus, such as a source of ultrasonic signals, transmits signals 110 into the patch or other delivery device, and at least one pad 14 or at least one layer of absorbent material, often through, but limited to, sonic membrane 11. Substance 15, contained within the at least one pad or at least one layer of absorbent material, is released in response to the impinging ultrasonic signals. The substance is then released from the at least one layer of absorbent material or the at least one absorbent pad and in some embodiments passes through semi-permeable membrane 13 and before being deposited on, in or through living tissue, which could be but is not limited to the surface of the patient's skin 3. While a delivery device has been described for use with ultrasound 110, other forms of external stimulus may be used in addition or in lieu of ultrasound. For example, iontophoresis, heat therapy, radio waves, magnetic transmission lasers, microwave signals, and/or electric currents applied to the living tissue, which in some embodiments is skin, may be used as the external stimulus. For example, ultrasonic signals may be used together with iontophoresis, or ultrasound may be used as a pre-treatment to the application of iontophoresis.

The terms “drug”, “medicinal compound”, “pharmaceutically active compound” and “pharmaceutical preparation”, as used herein, should be understood to be used in a non-limiting manner and for purposes of explanation only, as the present invention is suitable for delivering many substances to a patient. As used herein, the term “substance” may include, but is not limited to, any substance, solution or suspension including, but not limited to, a medicinal or non-medicinal substance which may be transported through a live surface or live membrane, including, but not limited to, live tissue and other types of live membranes. Such substances typically include one or more active ingredients and an excipient. “Excipient”, as used herein, generally refers to a substance used as a diluent or vehicle in a drug for the one or more active ingredients. Excipients are generally inert. Similarly, transdermally is used herein in a non-limiting manner, and includes intra-dermally (e.g., dermal delivery) and transmucosally as well, for example.

A pharmaceutical preparation and substance to be transdermally delivered is preferably in a liquid form. A liquid excipient is used as the carrier for the active substance. For example, a substance to be delivered transdermally may typically consist of an active substance suspended within a liquid carrier or adhesive.

By way of further example, active ingredients are generally immersed within an excipient binder fluid, such as saline or an acetate composition, to make them injectable. Insulin, for example, is often placed in acetate mixes. Common substance and pharmaceutical liquid carrier excipients include: water, distilled water, distilled water buffered, acetate, saline, phosphate, phosphate buffer with added protein, glycerin, saccharine, grapefruit aroma, methyl p-hydroxybenzoate, propyl p-hydroxybenzoate, sodium acetate, fructose, glucose or sucrose, hydrogenated glucose syrup, mannitol, maltitol, sorbitol, xylitol, gluten, tartrazine, arachis (peanut) oil, sesame oil, beeswax, benzyl alcohol, butylated hydroxyanisole, butylated hydroxytoluene, cetostearyl alcohol (including cetyl and stearyl alcohol), chlorocresol, edetic acid (edta), ethylenediamine, fragrances, hydroxybenzoates (parabens), imidurea, isopropyl palmitate, n-(3-chloroallyl)hexaminium chloride (quaternium 15), polysorbates, propylene glycol, sodium metabisulphite, wool fat and related substances including lanolin, and metacrystal solution.

Excipients typically include substituent components. For example, excipients may typically include one or more preservatives, like zinc, and one or more buffers, like dibasic sodium phosphate. Part of the present invention lies in the observation that certain substances and pharmaceutical preparations contain liquid carrier excipients, and/or excipient ingredients, that inhibit the mobility, and hence deliverability of the active ingredient(s) responsively to insonification. Part of the present invention lies in the observation that certain substances and pharmaceutical preparations contain liquid carrier excipients, and/or excipient ingredients, that increase the delivery rate of the active ingredient(s) responsively to insonification. Part of the present invention lies in the observation that certain substances and pharmaceutical preparations contain liquid carrier excipients, and/or excipient ingredients, that increase delivery volume per unit of time of the active ingredient(s) responsively to insonification.

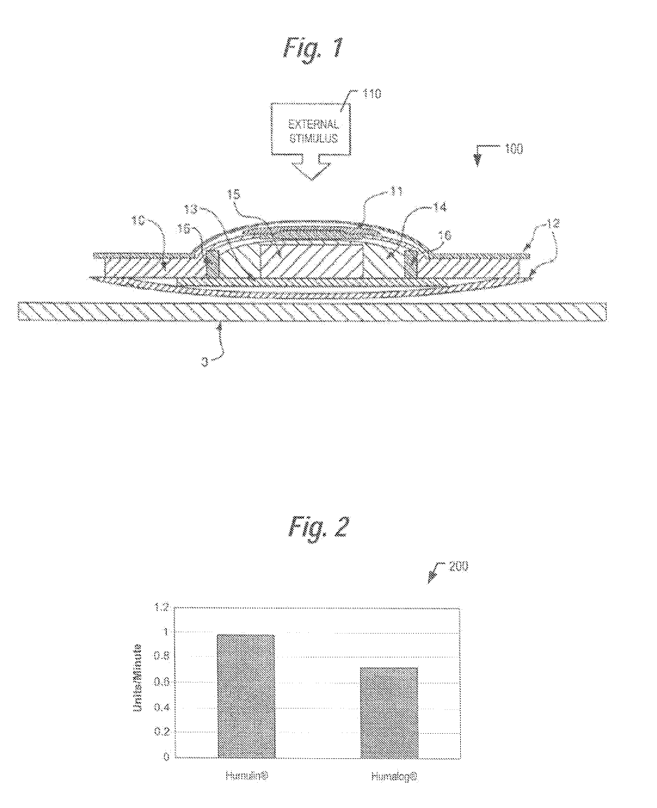

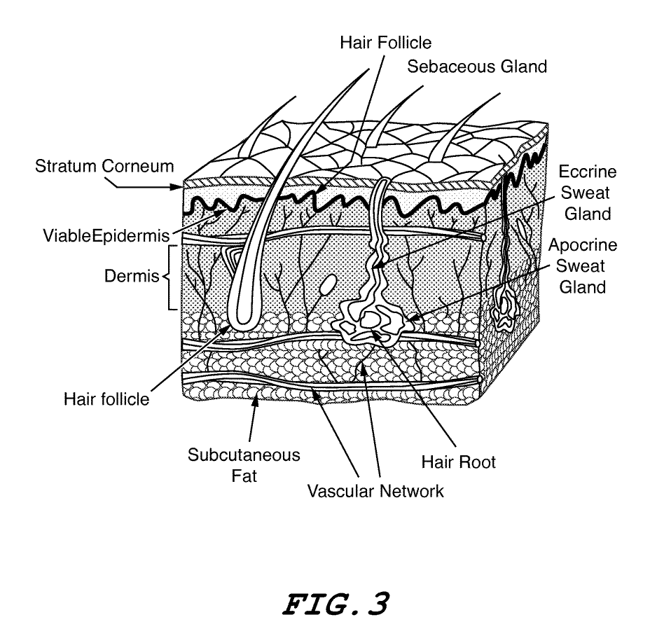

Referring now to FIG. 2, it shows a chart 200 indicative of human patient transdermal delivery rates for two commercially available insulin types responsively to insonification. The data of FIG. 2 is indicative of a transdermal delivery of u100 Humulin® and Humalog® to a human patient using a 125 mW, 30 kHz ultrasonic signal from a square, four-transducer array. Applicant have discovered that Humulin® is delivered at a faster rate and at a greater dose transdermally under ultrasonic propagation than Humalog®. This is due to changes in the chemistry between the two different versions of insulin. Humulin is designed as a slow acting basal insulin, Humalog is designed as a fast acting insulin, often prescribed before a meal.

Table-1 illustrates basic ingredients of Humulin® and Humalog®.

TABLE 1

| INGREDIENT | HUMULIN | HUMALOG |
|---|---|---|
| Biosynthetic human insulin | 100 units/ml | 100 units/ml |
| Glycerin | 16 mg | 16 mg |
| M-Cresol Preservative | 2.5 mg | 3.15 mg |
| Zinc Oxide | 0.017 mg | 0.0197 |
| Sodium Hydroxide + | Ph adjusted to 7.0 to 7.8 | Ph adjusted to |
| Hydrochloric acid buffer | | 7.0 to 7.8 |
| solution | | |
| Dibasic Sodium Phosphate | 0 | 1.88 g |
| Buffing and bulking agent | | |
| diluent | | |

In Table-1 it can be seen that the main difference between Humulin and Humalog insulin are: An increase in the amount of preservative, zinc oxide, a metallic preservative and an increase in the presence of dibasic sodium phosphate, phosphate is used as a buffering agent.

From FIG. 2 it is clear that the delivery rate of insulin varies between the Humalog® and Humulin® types of insulin, under ultrasonic propagation. Ultrasound interacts with metals and can excite the Zinc compound, leading to heating of the metallic ions.

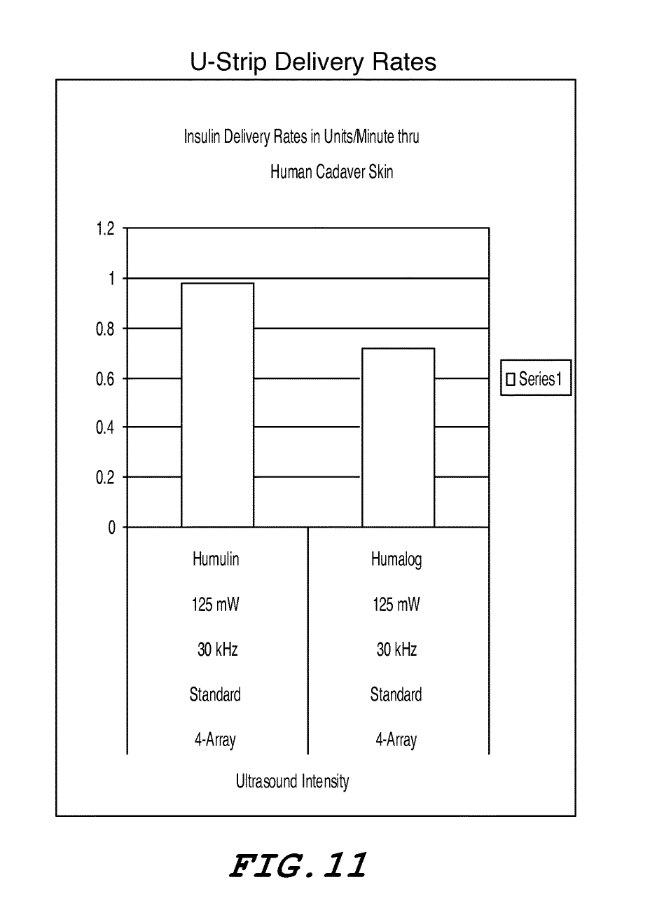

FIG. 2 shows a Comparison of the Delivery Rate of Humulin and Humalog Insulin at Similar Ultrasonic Frequency and Intensity Levels. A comparison of the delivery rate for Humulin and Humalog across the abdominal skin using a standard 4-element transducer array shows a 0.98-unit/minute-delivery rate for Humulin and a 0.72-unit/minute-delivery rate for Humalog insulin. The difference in delivery flow is due to the difference in interaction between the ultrasound and components within the target drug substance. There is a slight difference in the insulin loading and excipient and preservative package employed between Humulin and Humalog. The interaction of ultrasound with those ingredients could excite the overall pharmaceutical preparation at different driving rates, and therefore account for the faster or slower delivery of the drug across the skin. With all conditions the same, as shown in FIG. 11, the ultrasonic propagation through cadaver human abdominal skin was faster for Humulin than for Humalog.

Dibasic sodium phosphate has a specific gravity of 1.67 (higher than that of water, which is the bulk medium in the insulin variant, Humalog®). It is surmised this is the influencing factor that slows the delivery rate of Humalog® responsively to ultrasound.

By way of further, non-limiting example, ultrasound transduction through denser mediums is slower than through leaner or less dense materials. Ultrasound is reflected by dense materials and delivery power is impeded. It is surmised that the presence of high specific gravity dibasic sodium phosphate in Humalog® results in the impedance of ultrasound—thereby slowing transdermal delivery thereof.

### Experiment-1

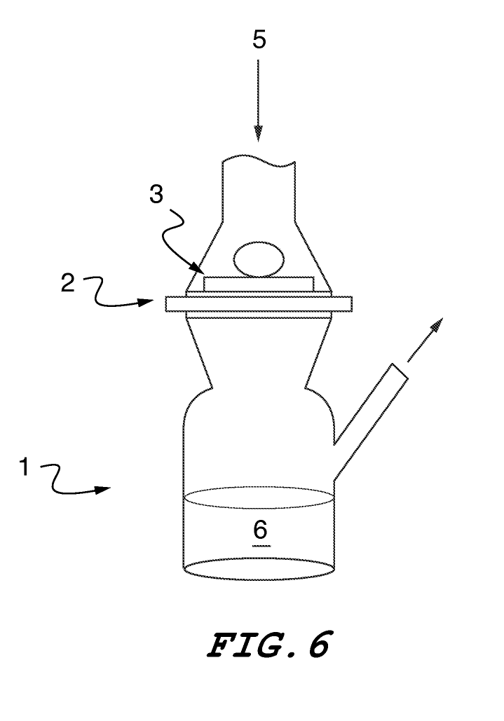

In Experiment-1 a sample of human whole cadaver abdominal skin was procured from Intermountain Tissue Center, sized to a 2.25″ diameter under an absorbent pad composed of Vicell-9009 cellulose at 1 millimeter thickness (6.3) and placed into a Franz cell (6.1) as illustrated in FIG. 6. An ultrasound source (6.5) is placed over the absorbent pad/cadaver skin sample (6.3) and clamped down onto the top flask (6.2).

A quantity of insulin, a standard 100 units, as measured by a standard insulin syringe is loaded onto the absorbent pad.

Next a thin film of polyethylene, 2.25 inch diameter by 0.156 millimeters thickness is placed a top of the absorbent pad/skin sample (6.3).

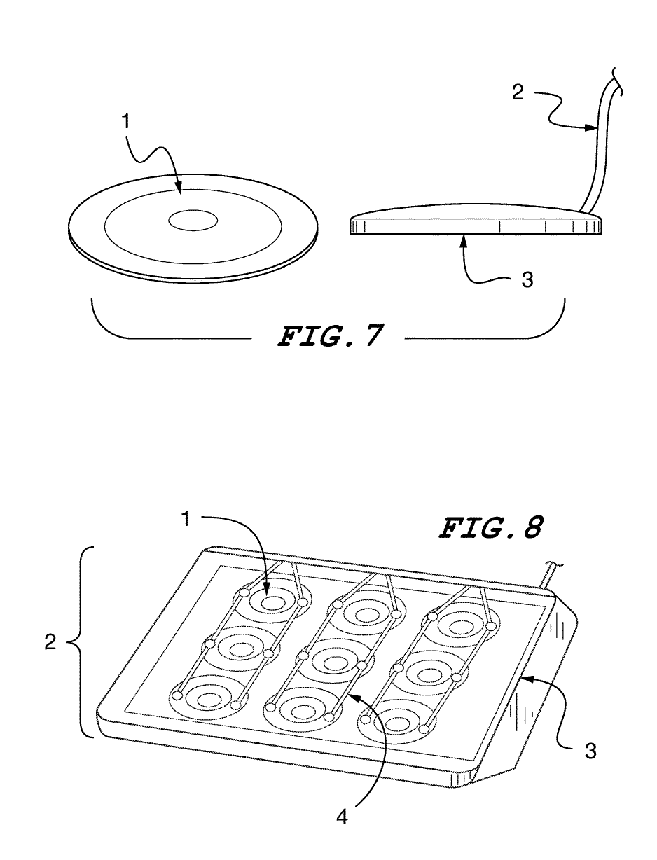

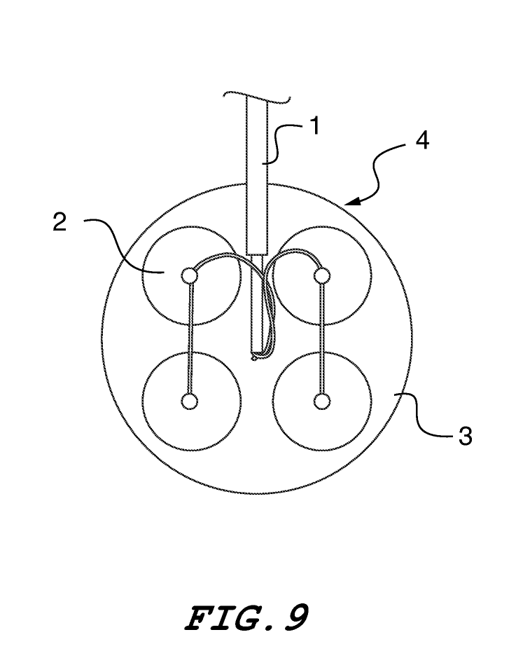

An ultrasound source (6.5) is placed onto the top portion of the flask (6.2) and may be either:
(1) a single element transducers as seen in FIG. 7,
(2) an array of 9 transducer elements as seen in FIG. 8,
(3) a standard array of 4 transducer elements affixed to a stainless steel face plate as seen in FIG. 9, or
(4) a stacked array of 4 transducer elements is affixed to a stainless steel face plate as seen in FIG. 9, with another layer of transducers placed atop the original layer.

Ultrasound drives insulin from the absorbent pad, and through the sample of human skin, and into the collection flask (6.4) where it collects (6.6). A sampling port (6.5) on the size of the cell (6.1) is used to draw samples of the collected drug (6.6) for analysis.

In experiment 1 a single element transducer as show in FIG. 7 was used to deliver insulin, 100 units of wither Humulin or 100 units of Humalog into the Franz cell. The single transducer element is a piezoelectric transducer crystal (7.1) designed to convert electrical energy into mechanical force, which was coated by an epoxy resin (7.3) and electrically connected to an ultrasonic generator circuit. The ultrasonic frequency was 23-30 KHz, at 125 mW/sq. cm intensity. Duration of ultrasonic excitation was 1 minute, but the experiment was conducted 10 times and an average delivery determined to be 14.7 units/hour for Humulin and 10.8 units/hour for Humalog.

Next a transducer system corresponding to the design shown in FIG. 9 was employed. In this configuration four piezoelectric crystals (9.2) are glued to a stainless steel face plate (9.3) using a conductive epoxy (9.4) and electrically connected so that all the crystals activate in tandem electrically through power sent through a cable (9.1). The ultrasonic frequency was 23-30 KHz, at 125 mW/sq. cm intensity for each transducer. The total output of power was 4×125=500 mW/sq. cm intensity

Using the transducer device of FIG. 9 the results were 58.8 units/hour for Humulin and 43.2 units/hour for Humalog.

Next a transducer system corresponding to the design shown in FIG. 8 was employed. In this configuration nine piezoelectric crystals (8.1) are fixed within a polymer potting panel (8.3) and electrically connected so that all the crystals activate in tandem electrically through power sent through a cable (8.4). The Array of transducers (8.2) is connected to an ultrasonic generator circuit. The ultrasonic frequency was 23-30 KHz, at 125 mW/sq. cm intensity for each transducer. The total output of power was 9×125=1,125 mW/sq. cm intensity

Using the transducer device of FIG. 9 the results were 105.8 units/hour for Humulin and 77.76 units/hour for Humalog.

Next a transducer system corresponding to the design shown in FIG. 9, was employed but in this configuration there was another layer of four transducer elements placed over top of the original ones affixed to the face plate. This configuration used eight piezoelectric crystals, four on top of the bottom layer. The ultrasonic frequency was 23-30 KHz, at 225 mW/sq. cm intensity for each transducer. The total output of power was 4×225=900 mW/sq. cm intensity

Using the stacked transducer device of FIG. 9 the results were 70.56 units/hour for Humulin and 51.84 units/hour for Humalog.

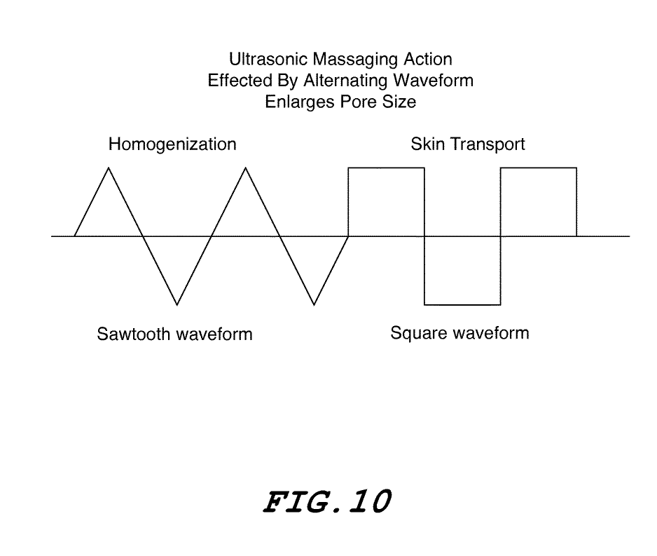

The ultrasonic transmission used in these experiments is illustrated in FIG. 10 wherein a saw tooth waveform lasting for 50 milliseconds is immediately followed by a square wave form of like 50 millisecond duration. This alternation between ultrasonic waveforms helped to prevent cavitation or heat within the drug.

These findings are corroborated by Table-2, which indicates that Humalog® is propagated at a slower rate of delivery than delivery rate for Humulin®. It is surmised that the presence of denser dibasic sodium phosphate in Humalog® enables it to “elute” or diffuse through the skin faster than Humulin®, which lacks the same.

TABLE 2

| Insulin Type | Humulin ® | Humalog ® |
|---|---|---|
| Using Single element | 14.7 units/hour | 10.8 units/hour |
| transducer(See FIG. 7) | | |
| Standard transducer Array | 58.8 units/hour | 43.2 units/hour |
| consisting of 4 transducers | | |
| (See FIG. 9) | | |
| Standard transducer Array | 105.8 units/hour | 77.76 units/hour |
| consisting of 9 transducers | | |
| (See FIG. 8) | | |
| Stacked transducer Array, 4 | 70.56 units/hour | 51.84 units/hour |
| transducers stacked on top of | | |
| 4 standard transducers (See | | |
| FIG. 9 with second rack of | | |
| transducers) | | |

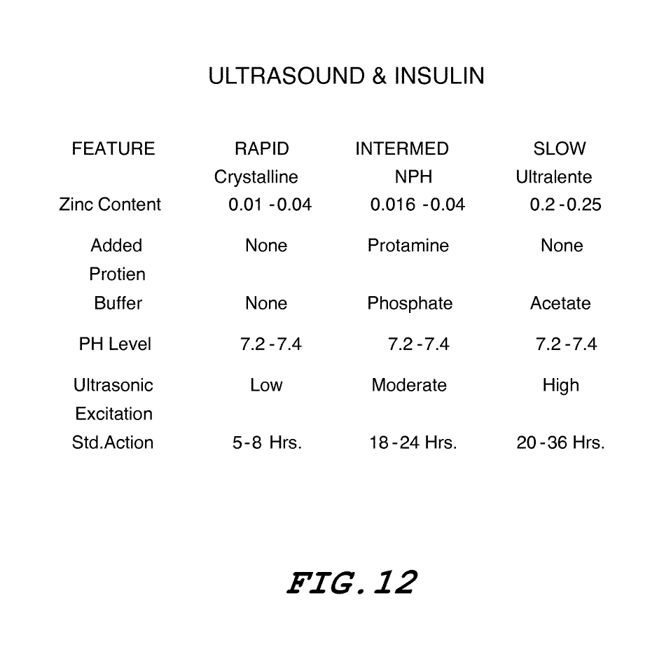

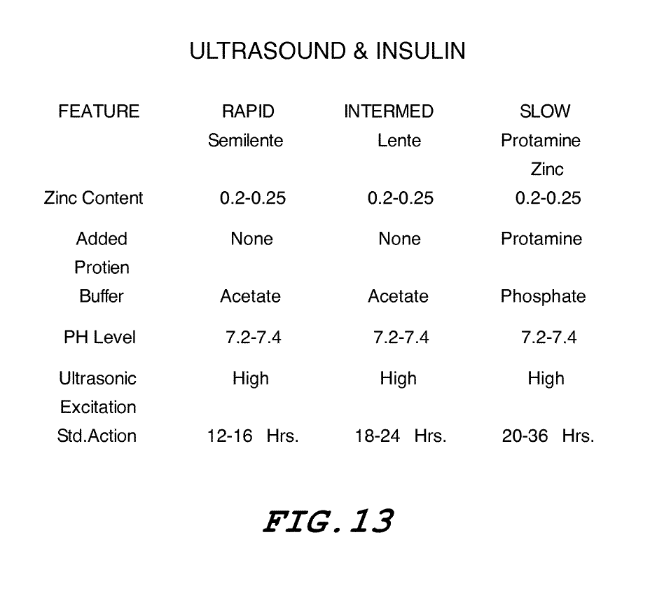

Examination of additional insulin formulations shows slower or faster delivery rates. FIGS. 12 and 13 contain a table of those observations.

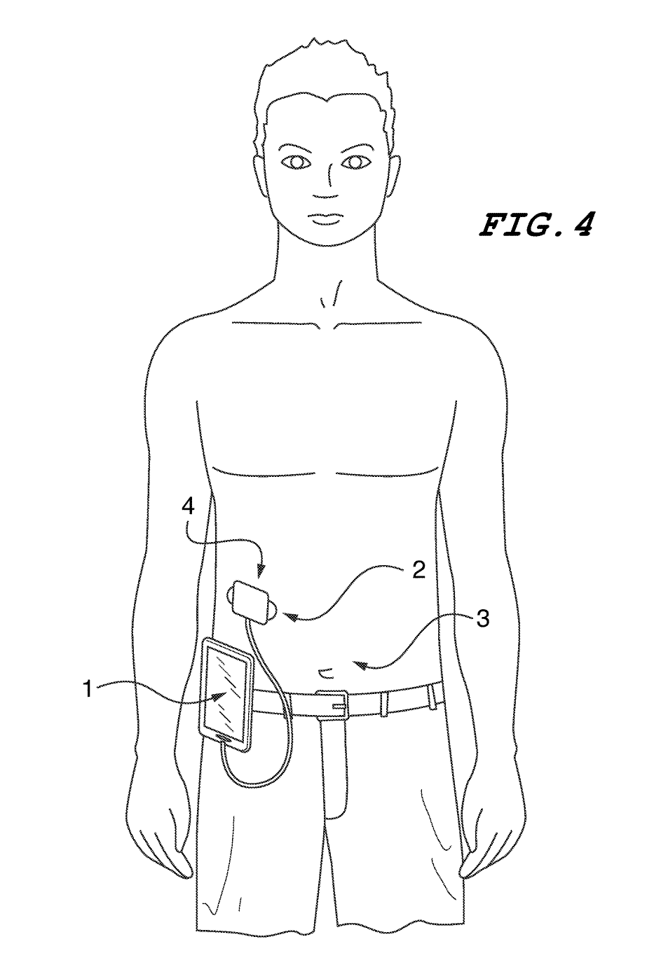

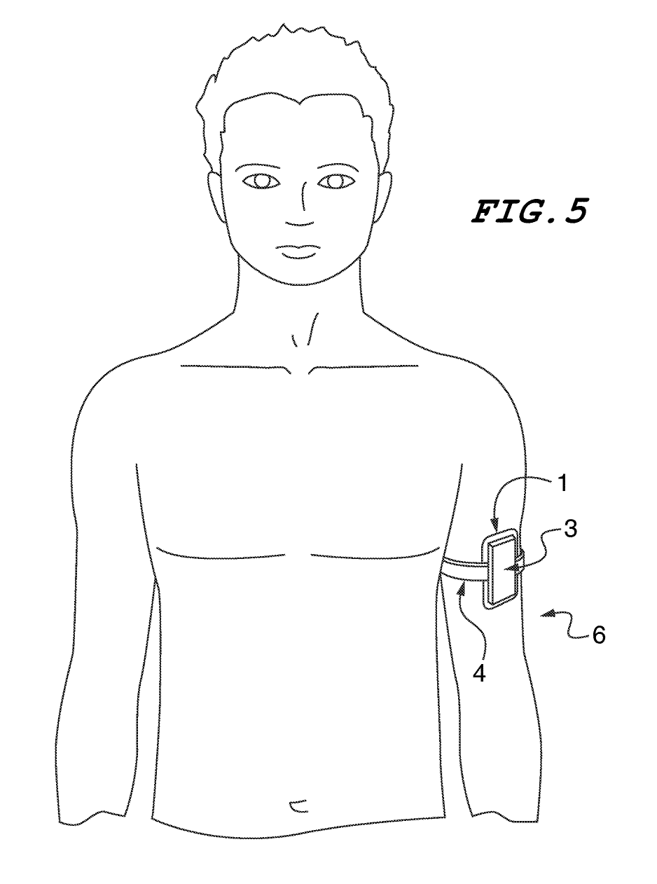

The observation of different delivery rates for differing insulin formulations shows that an ultrasonic transdermal drug delivery system, as shown in FIG. 4 and FIG. 5, may need to be programmed based upon the delivery rate of the insulin used in conjunction with a standard ultrasonic signal generation.

In FIG. 4, a waist mounted ultrasonic delivery system is shown, where a modified transdermal patch (4.3) is loaded with a target drug, in this instance insulin, and mounted onto the abdomen of a patient (4.4) A controller device (4.1) delivers electrical signals to the transducer coupler (4.2) which forces insulin from the patch into the skin of the patient, effecting ultrasonic insulin delivery.

In FIG. 5, an arm mounted ultrasonic delivery system is shown, where a modified transdermal patch (5.3) is loaded with a target drug, in this instance insulin, and mounted onto the arm of a patient (5.6) A controller device (5.1) is fitted on, or near or over the patch (5.3) and held in place by an arm band (5.4). The control unit (5.1) delivers electrical signals to the transducer coupler connected to the patch (5.3), which forces insulin from the patch into the skin of the patient, effecting ultrasonic insulin delivery.

### CONCLUSION

Basically, insonification changes the delivery equation in relation to the excipient formulation used in the drug carrier.

Further, dibasic sodium phosphate is a source for sodium ions in Humalog®. Sodium ions are positively charged. The presence of excess positive charges may lead to interactions with oppositely charged materials in a substance containing patch—and adsorb or absorb therein or thereto via weak electrostatic attraction. This may obstruct the path of substance, e.g., insulin, molecules through the solution from the patch to the skin, and there-through. Alternatively, excess positive charges may interact with the substance, e.g., insulin, molecules, themselves, which then adsorb or absorb onto patch materials, or even the biotransformable layers of the skin, thereby slowing their delivery into viable skin or blood circulation.

Regardless, ultrasonically induced transdermal delivery of an insulin and dibasic sodium phosphate containing substance from a patch, which may incorporate an absorbent material (FIG. 1), may be hastened by reducing the amount of dibasic sodium phosphate therein. It is surmised that ultrasonically induced transdermal delivery of other substances, such as other pharmaceutical preparations including other active ingredients, can be similarly improved by reducing the amount of dibasic sodium phosphate therein or altering the ingredient composition of the excipient solution with less metals, and less preservative. Similarly, it is surmised that ultrasonically induced transdermal delivery of substances, such as insulin containing drugs, can be improved by reducing the amount of excipient ingredients having a specific gravity greater than that of water. Similarly, it is also surmised that ultrasonically induced transdermal delivery of substances, such as insulin containing drugs, can be improved by predominantly using excipient ingredients having a specific gravity less than 1.67.

It should be understood that just as medications and other substances are made using conventional processes, re-formulated drugs and substances that have a reduced amount of dibasic sodium phosphate, a reduced amount of excipient ingredients having a specific gravity greater than that of water, and/or predominantly use excipient ingredients having a specific gravity less than 1.67 may be similarly processed.

According to an aspect of the present invention, substances can be identified as good candidates for ultrasonic transdermal delivery or re-formulation for transdermal delivery using similar criteria. For example, a list of substances and corresponding compositions may be made or acquired. Those substances having excipient ingredients with specific gravities under 1.67 and/or omitting dibasic sodium phosphate and/or having excipient components with specific gravities around that of water may be selected as good candidates for ultrasonically induced transdermal delivery. Similarly, those substances having excipient ingredients with specific gravities around 1.67 or higher, and/or including dibasic sodium phosphate may be selected as less attractive candidates for reformulation for ultrasonically induced transdermal delivery. The good candidates are predicted to generally have a higher ultrasonically induced transdermal delivery rate of the active ingredient(s) thereof higher than less attractive candidates have the same active ingredient(s). By way of further, non-limiting example only, Humulin® may be classified in a first class as being a good candidate, while Humalog® may be classified in a second class as being a less attractive candidate for rapid transdermal delivery from a patch responsively to ultrasound insonification.

Part of the present invention also lies in the observation that certain substances and pharmaceutical preparations containing liquid carrier excipients, and/or excipient ingredients that include sodium, such as but not limited to saline, increase the delivery rate of the active ingredient(s) responsively to insonification and enable the delivery of larger compounds responsively to insonification. Part of the present invention lies in the observation that certain substances and pharmaceutical preparations containing liquid carrier excipients, and/or excipient ingredients that include dibasic sodium phosphate increase the delivery volume per unit of time of the active ingredient(s) responsively to insonification and enable the delivery of larger compounds responsively to insonification.

While this application has used variations between differing insulin's it should be noted that other drug products intended for ultrasonic transdermal drug delivery will similarly need to be tested for their ultrasonic dose propagation properties. Indeed this methodology can be utilized to re-formulate a drug to make it more susceptible to ultrasonic propagation, by re-formulating the excipient carrier component.

An embodiment of the invention, is a method for improving the transdermal delivery rate of an active ingredient in a substance made in accordance with a dibasic sodium phosphate containing formulation responsively to insonification thereof, comprising: reducing the amount of dibasic sodium phosphate in the formulation to provide a reduced dibasic sodium phosphate formulation; and, making a substance in accordance with the reduced dibasic sodium phosphate formulation. In some embodiments, the substance further comprises insulin. In some embodiments, the insonifying comprises ultrasonic insonification. In some embodiments, the reducing the amount of dibasic sodium phosphate comprises replacing the dibasic sodium phosphate with a component having a specific gravity less than 1.67. In some embodiments, the reducing comprises replacing said dibasic sodium phosphate with a component having a specific gravity approximately that of water.

An embodiment of the invention is a method for classifying the predicted suitability of a substance for ultrasonically induced transdermal delivery into a patient comprising: a) determining if the substance contains dibasic sodium phosphate; and, b) if it does, classifying the substance in a second class; and c) if it does not, classifying the substance in a first class, and d) wherein, the first class is associated with substances having a relatively high predicted transdermal delivery rate, and the second class is associated with substances having a relatively low predicted transdermal delivery rate. In some embodiments, a list of substances are provided, wherein the above-referenced determining and classifying occurs for each of the substances on list.

An embodiment of the invention is a method for improving the transdermal delivery of an active ingredient in a substance by varying the excipient solution or carrier accompanying said active ingredient, to enable a greater speed of transdermal delivery when used with an ultrasonic drug delivery system.

An embodiment of the invention is an ultrasonic delivery system for delivering a drug, which employs a transdermal patch containing at least one absorbent pad, and which is connected to either a single or array of transducers, and controlled by a wearable control device, wherein ultrasound is delivered through the drug laden patch and delivers the dose to the patient. In some embodiments, the system is mounted on the arm of the patient. In some embodiments, the system is mounted on the waist of the patient. In some embodiments, the ultrasonic transmission may be a standard sinusoidal waveform version of ultrasonic transmission. In some embodiments, the ultrasonic transmission may be a combination of ultrasonic waveforms.

An embodiment of the invention is an ultrasonic delivery system for delivering a drug, which employs a transdermal patch containing at least one absorbent pad, and which is connected to either a single or array of transducers, and controlled by a wearable control device, wherein ultrasound is delivered through the drug laden patch and delivers the dose to the patient, wherein a alternating ultrasonic waveform transmission is employed to avoid cavitation or over heating the drug or the patients skin.

It will be apparent to those skilled in the art that modifications and variations may be made in the apparatus and process of the present invention without departing from the spirit or scope of the invention. It is intended that the present invention cover the modification and variations of this invention provided they come within the scope of the appended claims and their equivalents.

What is claimed is:

1. A method for improving transdermal delivery of an active ingredient in a substance made in accordance with a dibasic sodium phosphate containing formulation responsively to insonification thereof, comprising:
a) Reducing the amount of dibasic sodium phosphate in the formulation to provide a reduced dibasic sodium phosphate formulation; and,
b) Making a substance in accordance with the reduced dibasic sodium phosphate formulation.

2. The method of claim 1, wherein said substance further comprises insulin.

3. The method of claim 2, wherein said insonifying comprises ultrasonic insonification.

4. The method of claim 1, wherein said reducing comprises replacing said dibasic sodium phosphate with a component having a specific gravity less than 1.67.

5. The method of claim 1, wherein said reducing comprises replacing said dibasic sodium phosphate with a component having a specific gravity approximately that of water.

6. A method for improving transdermal delivery of an active ingredient in a substance made in accordance with an excipient containing formulation, wherein said excipient includes at least one component having a specific gravity of at least around 1.67, responsively to insonification thereof, comprising:
a) Reducing the amount of excipient component having a specific gravity of at least around 1.67 to provide a second formulation; and,
b) Making a substance in accordance with the second formulation.

7. The method of claim 6, wherein said at least one active ingredient comprises insulin.

8. The method of claim 7, wherein said insonification comprises ultrasonic insonification.

9. The method of claim 7, wherein said reducing comprises replacing said excipient component having a specific gravity of at least around 1.67 with a component having a specific gravity less than 1.67.

10. The method of claim 7, wherein said reducing comprises replacing said excipient component having a specific gravity of at least around 1.67 with a component having a specific gravity approximately that of water.

11. A method for classifying the predicted suitability of a substance for ultrasonically induced transdermal delivery into a patient comprising:
a) Determining if the substance contains dibasic sodium phosphate; and,
b) If it does, classifying the substance in a second class; and,
c) If it does not, classifying the substance in a first class;
d) Wherein, said first class is associated with substances having a relatively high predicted transdermal delivery rate, and said second class is associated with substances having a relatively low predicted transdermal delivery rate.

12. The method of claim 11, further comprising providing a list of substances, wherein said determining and classifying occurs for each of said substances on said list.

13. A method for improving the transdermal delivery of an active ingredient in a substance by varying the excipient solution or carrier accompanying said active ingredient, to enable a greater speed of transdermal delivery when used with an ultrasonic drug delivery system.

14. An ultrasonic delivery system for delivering a drug, which employs a transdermal patch containing an absorbent pad, and which is connected to either a single or array of transducers, and controlled by a wearable control device, wherein ultrasound is delivered through the drug laden patch and delivers the dose to the patient.

15. An ultrasonic delivery system for delivering a drug, according to claim 14, which is mounted on the arm of the patient.

16. An ultrasonic delivery system for delivering a drug, according to claim 14, which is mounted on the waist of the patient.

17. An ultrasonic delivery system for delivering a drug, according to claim 14, where the ultrasonic transmission may be a standard sinusoidal waveform version of ultrasonic transmission.

18. An ultrasonic delivery system for delivering a drug, according to claim 14, where the ultrasonic transmission may be a combination of ultrasonic waveforms.

19. An ultrasonic delivery system for delivering a drug, which employs a transdermal patch containing an absorbent pad, and which is connected to either a single or array of transducers, and controlled by a wearable control device, wherein ultrasound is delivered through the drug laden patch and delivers the dose to the patient, wherein a alternating ultrasonic waveform transmission is employed to avoid cavitation or over heating the drug or the patients skin.
